SEQUENCE LISTING (12 sequences)

SEQ ID NO: 1 - protein, 6 aa, Homo sapiens
  misc_feature (4)..(5): Xaa can be any naturally occurring amino acid
DSGXXS

SEQ ID NO: 2 - DNA, 26 bp, Artificial
  oligonucleotide primer
cgggatccatggataaagtccatacc

SEQ ID NO: 3 - DNA, 27 bp, Artificial
  oligonuceotide primer
cccaagcttttagagaagcatcaaatg

SEQ ID NO: 4 - DNA, 27 bp, Artificial
  oligonucleotide primer
gcgggatccatggataaagtccatacc

SEQ ID NO: 5 - DNA, 27 bp, Artificial
  oligonucleotide primer
cccaagcttttagagaagcatcaaatg

SEQ ID NO: 6 - DNA, 30 bp, Artificial
  oligonucleotide primer
cctttgatattggatcctaagcttttagag

SEQ ID NO: 7 - DNA, 45 bp, Artificial
  oligonucleotide primer
cggtgggggaggcggtgggggaggcggatccatggatagagtcca

SEQ ID NO: 8 - DNA, 36 bp, Artificial
  oligonucleotide primer
catctctagaacctgcagggaatgcagatcttcgtg

SEQ ID NO: 9 - DNA, 45 bp, Artificial
  oligonucleotide primer
ccccaccgcctcccccaccgcctccctcgagacggccgcccctca

SEQ ID NO: 10 - DNA, 24 bp, Artificial
  oligonucleotide primer
gggccgctctaaaacccgcaggga

SEQ ID NO: 11 - DNA, 24 bp, Artificial
  oligonucleotide primer
aagcctctagagaagcatcacaat

SEQ ID NO: 12 - protein, 1349 aa, Homo sapiens
ETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDLGSNMDPAEAVLQEKALKFMNSSEREDCNNGEPPRKIIPEKNSLRQTYNSCARLCINQETVCLTSTAMKTENCVAKAKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDENAPPNSFYRALYPKIIQDIETIESNWRCGRHSLQRIHCRSETSKGVYCLQYDDQKIVSGLRDNTIKIWDKSTLECKRILTGHTGSVLCLQYDERVIITGSSDSTVRVWDVNAGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLMAALDPRAPAGTLCLRTLVEHSGRVFRLQFDEFQIVSSSHDDTILIWDFLNDPAAHAEPPRSPSRTYTYISRGSNPTLMSVPQYRPRPANPDEIGNFIDENLKAADSDPTAPPYDSLLVFDYEGSGSEAASLSSLNSSESDQDQDYDYLNEWGNRFKKLADMYGGGED

(19) United States
(12) Patent Application Publication
(10) Pub. No.: US 2008/0139467 A1
(43) Pub. Date: Jun. 12, 2008

(54) Method and Products For the Selective Degradation of Proteins

(76) Inventors: John Colyer, Leeds (GB); Moninder Singh Bhogal, Leeds (GB)

Correspondence Address: MYERS BIGEL SIBLEY & SAJOVEC, PO BOX 37428, RALEIGH, NC, 27627, US

(21) Appl. No.: 10/591,109

(22) PCT Filed: Mar. 3, 2005
(86) PCT No.: PCT/GB05/00811

§ 371 Date: Oct. 5, 2007

(30) Foreign Application Priority Data
Mar. 3, 2004 (GB) 0404731.2

(51) Int. Cl. A61K 38/00; C12N 5/06; C07K 14/00; C12N 9/50; A61P 9/00; C12N 15/11; C12N 15/00; A61K 31/70

(52) U.S. Cl. 514/12; 435/375; 530/350; 435/219; 536/23.1; 435/320.1; 435/325; 514/44; 536/25.3

(57) ABSTRACT

The present invention provides a method of controlling levels/concentrations of a target moiety. The method of the present invention includes introducing into a cell a product comprising at least one of each of the following modules: a targeting module, a destruction module and a replacement module. The method further comprises targeting the target moiety with the targeting module so that the destruction module can effect degradation of the target moiety and replacing the target moiety with a replacement: module. The replacement module may be either a modified form of the target moiety itself or a functional unit which restores normal metabolic activity of the cell. The present invention also a destruction module and a replacement module, nucleic acids encoding the products and vectors. The present invention also provides for use of the method and products in treating various clinical disorders.

The present invention relates to a method of, and products for, manipulating the concentration or level of a target cellular protein by introducing into a cell, a modified form of the protein, the modified form having the ability to target and alter the degradation of the target cellular protein and subsequent replacement of the target protein with either the modified form of the protein or a replacement module. The present invention also provides a modified form of phospholamban (PLB), production and uses thereof, for use particularly but not exclusively, in the treatment of calcium homeostasis abnormalities and especially in the treatment of myocardial disorders. The present invention also provides a formulation and pharmaceutical comprising the modified form of PLB for the treatment of calcium homeostasis abnormalities and particularly heart failure.

### BACKGROUND TO THE INVENTION

It is known that proteins are in a constant state of flux, being continually synthesised and degraded. The two principle routes of protein degradation are the secretory pathway (transport through the Golgi network to the lysosomal compartment) and the proteasomal route (a regulated pathway involving “tagging” by ubiquitin followed by recognition and degradation by the proteasome).

A number of methods of removing proteins from their native cellular environments have been reported. For example, by catalytic antagonists (CA) which can be defined as enzymes that destroy protein function. CAs consist of a ligand binding or targeting motif domain, which confers specificity to a target protein so that it binds the target at a functionally relevant site so bringing a proteinase domain or destruction motif into close proximity of the target protein that is able to degrade a broad range of substrates. Davis et al¹ have used simple model systems to test the CA hypothesis, employing avidin/biotin binding partners. Avidin is a tetrameric protein complex of 66 kDa and biotin is small molecule (224 Da) that binds to avidin with an extremely high affinity (K_{d}: 1×10⁻¹⁵M). The protease subtilisin, which has a broad substrate specificity was attached to biotin (targeting ligand) by first introducing a cysteine residue at position 156 (site directed mutagenesis) and secondly reacting this in the presence of methanethiolsulphate (MTS) in order to attach biotin. Thus binding of biotin to subtilisin produces a chimeric proteinase or CA. This molecule was shown to selectively degrade avidin with a 345-fold increase in specificity over free subtilisin.

Another method of removing proteins from their native cellular environment is “engineered proteolysis” which is currently being developed particularly as an anti-cancer strategy. In this instance, substrate specificity is engineered into a component of the degradation machinery (F-box protein of an E3 ubiquitin ligase), which is then able to recruit the target protein for ubiquitination and subsequent degradation by the proteasome. An example utilizing this approach is β-catenin, a number of human cancers have been associated with aberrant β-catenin signalling. It appears that mutations in the N-terminal domain of β-catenin that compromise phosphorylation lead to uncontrolled increase in β-catenin and therefore gene transcription leading ultimately to tumourigenesis. The concentration of β-catenin in the cell is maintained at a low steady state level by regulated proteolysis. In the absence of an extracellular signal (wnt signaling pathway) β-catenin forms a destruction complex with glycogen synthase-3-kinase (GSK3), axin and adenomatosis polyposis coli (APC). In this complex β-catenin is phosphorylated in the consensus motif, DSGXXS (SEQ ID NO:1), found at the N-terminal domain. β-catenin is phosphorylated at this consensus sequence and forms a substrate for the F-box protein β-TrcP, which binds β-catenin and recruits it to the SCF E3 ubiquitin ligase for ubiquitination and subsequent degradation by the proteasome. However, in the presence of wnt signalling the destruction complex falls apart and β-catenin is no longer phosphorylated at this critical site. The dephosphorylated form of β-catenin is no longer a substrate for the F-box protein and thus an increase in the concentration of β-catenin eventually results in its movement into the nucleus, where it initiates transcription of β-catenin target genes.

The invention described hereinafter improves upon the prior art degradation strategies and has been particularly exemplified with respect to phospholamban proteins.

Phospholamban (PLB) is a small membrane protein located in the sarcoplasmic reticulum (SR) of cardiac myocytes and is a potent regulator of myocardial contractility. Underlying this regulation is the ability of PLB, whilst dephosphorylated, to associate with Ca²⁺ ATPase (SERCA2a) and inhibit Ca²⁺-pump activity. This reduces the rate of Ca²⁺-sequestration by the SR, which in turn slows the rate of relaxation and reduces the force of subsequent cardiac muscle contractions. These events are reversed upon phosphorylation of PLB on one of at least two sites. Although this regulatory system has many advantages in the healthy heart, not least establishing and controlling access to the substantial cardiac reserve, under conditions of heart failure it becomes problematic.

In a number of cardiac disorders such as acute congestive heart failure precipitated by mycordial ischemia, hypertrophic cardiomyopathy and dilated cardiomyopathy the underlying abnormality appears to be diastolic dysfunction, i.e. lethargic of Ca²⁺ handling by the SR. As a result of low Ca²⁺-pump activity there is abnormally slow relaxation of the heart muscle and low levels of force generated upon each contraction. Accordingly, modulation of Ca²⁺ cycling as a therapeutic strategy in the management of heart failure has become a relevant target and a variety of diverse approaches to improve contractility have been proposed.

It is known that a number of factors contribute to low Ca²⁺-pump activity, one of these is the reduced level of expression of SERCA2a leading to reduced Ca²⁺-transport rates. Another factor is the enhanced inhibition of SERCA2a by PLB due to an increase in the stoichiometry of PLB with respect to SERCA and also to a reduction in the phosphorylation status of phospholamban, which increases the concentration of the inhibitory species.

Antagonism of the effect of PLB, either by antibody binding to the protein or by ablation of the gene for PLB causes a dramatic increase in the kinetics of Ca²⁺-handling and the force of muscle contraction. These appear to be desirable changes, which are without adverse consequence in the healthy heart (mouse) and which might correct the dominant clinical features of heart failure. In animal models, antagonism of the PLB:SERCA2a interaction has indeed improved the clinical status of mice and hamsters with experimental forms of heart failure. In a dilated cardiomyopathy model, generated by the deletion of muscle specific LIM domain cytoskeletal protein (MLP)², heart failure is apparent in the neonate and increases progressively in severity thereafter. Ablation of PLB in this mouse (knockout for MLP and PLB) prevents the development of heart failure to 6 months (the time limit of the study), which underlines the therapeutic potential of PLB ablation. In a second study, the delivery by gene therapy of a dominant negative form of PLB (S16E mutant), which mimicks the phosphorylated form of PLB and can be termed a pseudo-phosphorylated form of PLB, into the heart of cardiomyopathic hamsters acts as an antagonist to wild type PLB (by simple mass action). Antagonism of PLB in this way prevents the development of heart failure by restoring normal Ca²⁺-handling by the SR³.

The therapeutic potential of PLB ablation or antagonism has been demonstrated from these animal studies. Extrapolation to man however is not straightforward or immediately predictable in that, two point mutations in the PLB gene, have been discovered in the human population; both of these alter the PLB protein sequence and are associated with heart failure^{4,5}. The effects of one of these mutations can be explained using the dogma supported by animal studies, whereas the confusion and unpredictability is that the other cannot. In the case of the first point mutation, R9C, the mutant form of PLB appears to accentuate the inhibitory potential of wild type PLB in heterozygous individuals by suppressing phosphorylation of wild type PLB. This increases Ca²⁺-pump inhibition which, as has been shown in animal studies, is associated with heart failure. The second mutation however, L37stop, results in the production of a truncated PLB protein which is unstable. The steady state concentration of PLB in these individuals is low (undetectable), but, in contrast with the (healthy) phenotype this produces in mice, the absence of PLB in man is associated with heart failure⁴. This result is unexpected from the observations in animal models, and suggests that complete antagonism of PLB would be undesirable in the treatment of heart failure. These studies suggest that a fine balance in Ca²⁺-handling must be maintained to avoid heart failure. Deviation from these ‘normal’ limits, in either direction (hypercontractility (PLB ablation⁵) or hypocontractility (dominant PLB), prompts adaptive changes leading to heart failure.

The present invention provides a unique approach to targeting and selective degradation of a cellular protein or other moiety and subsequent replacement thereof with a replacement module, in this way the prior art methods of CA and engineered proteolysis can be extended to include a designer replacement feature. In particular, the present invention provides for the removal of PLB from a biological system and an alternative approach to targeting Ca²⁺cycling for the management of heart failure. The present invention provides alternative therapeutics for the treatment of heart failure, such therapeutics could offer immediate benefit to sufferers of heart failure/disease in addition to providing models to study the complex interactions of the Ca²⁺-handling process which will be of benefit to the pharmaceutical industry in designing target drugs/therapeutics.

### STATEMENT OF THE INVENTION

The present invention provides a method and products for targeting cellular proteins in general, provoking their degradation from their native cellular environment and their subsequent replacement with either a modified or mutated form of the protein itself or with a replacement module, the replacement entity retaining the beneficial qualities of the target entity. The invention discloses a modified or mutated form of a PLB nucleic acid and protein encoded thereby, the modified or mutated form of the wild type protein being modified so that the modified or mutated PLB contains information altering the degradation rate and process of its partner polypeptides, namely wild type PLB. The modified or mutated protein is resistant to ubiquitination. The modified or mutated form of PLB nucleic acid or protein encoded thereby is conveniently referred to as vitiate PLB (vPLB) and the protein encoded by the nucleic acid is designed so as to stimulate catalytic removal of wild type PLB protein from cells. In addition the vPLB can be further modified to retain beneficial qualities of the wtPLB and exclude those which may be detrimental.

Throughout the specification and the claims which follow, unless the context requires otherwise, the word “comprise”, or variations such as “comprises” or “comprising” will be understood to imply the inclusion of a stated integer or group of integers but not the exclusion of any other integer or group of integers.

According to a first aspect of the invention there is provided a method of controlling levels/concentrations of a target moiety comprising:
(i) causing or allowing the introduction into a cell a product comprising at least one of each of the following modules: a targeting module, a destruction module and a replacement module;
(ii) causing or allowing the targeting of the target moiety with the targeting module of the product so as to bind them together or at least bring the target moiety into close proximity with the product so that the destruction module can effect degradation of the target moiety; and
(iii) causing or allowing the replacement of the target moiety with a replacement module, the replacement module either being a modified form of the target moiety itself or a functional unit which restores normal metabolic activity of the cell.

In other words, the invention includes a process in which said product targets the targeting moiety whereby said product and said targeting moiety come into relative locations at which the destruction module can degrade the target moiety. The target moiety may then be replaced by the replacement module. The process may occur in a cell.

Reference herein to a targeting module is interchangeable with the terms “targeting motif” or “targeting domain” or “targeting ligand” and is intended to include a protein or polypeptide or aptamer or drug-like molecule or portions or fragments thereof. The targeting module of the product confers specificity of the product for the target moiety.

Reference herein to a destruction module is interchangeable with the terms “degradation module”, “destruction/degradation motif” or “destruction/degradation domain” and is intended to include a protein or polypeptide or aptamer or portions or fragments thereof. The destruction module of the product is able to direct degradation of the target moiety.

Reference herein to a replacement module is interchangeable with the terms “replacement motif” or “replacement domain” and is intended to include a protein or polypeptide or aptamer or portions or fragments thereof. The replacement module of the product may be in the form of a modified form of the target moiety itself or in the form of a functional unit which is capable of restoring normal metabolic activity of the cell into which the product has been introduced.

Preferably, the destruction module may act directly on the target moiety such as when the module comprises a proteinase or it may act indirectly on the target moiety such as when the module comprises a mutation or deletion such that it alters degradation of the target moiety by for example redirecting the normal degradation pathway.

Preferably, the replacement module may be further modified to retain beneficial attributes and to remove/delete any adverse attributes. Ideally the replacement module is able to restore normal metabolic activity.

It will be appreciated that the method of the present invention advantageously provides a single product for both removal and replacement of the target moiety which improves on the prior art which has hitherto been restricted to only elimination.

According to a further aspect of the invention there is provided a product comprising at least one of each of the following modules: a targeting module, a destruction module and a replacement module as hereinbefore described. It will be appreciated the product may contain more than one of each component.

In one embodiment of the invention the product comprises a targeting module comprising a β-catenin binding domain of E-cadherin, a destruction module comprising a mutated F-box such that it is not able to bind to the phosphorylated form of β-catenin and a replacement module comprising wild type β-catenin. It will be appreciated that the product in this embodiment indirectly affects degradation of the target moiety i.e. mutant (cancerous) β-catenin.

In a further embodiment of the invention the product comprises a targeting module comprising an inhibitor of alcohol dehydrogenase (ADH), a destruction module comprising a protease domain and a replacement module comprising a functional ADH with any protease recognition motifs removed and, typically, specific mutations to reduce substantially sensitivity to the targeting module (which is also an ADH inhibitor). It will be appreciated that the product in this embodiment directly affects degradation of the target moiety i.e. ADH.

Preferably the inhibitor of ADH is 4-hexylpyrazole.

In a yet further embodiment of the invention the product comprises a targeting module comprising a PLB target motif, a destruction module comprising absence of either or both lysine 3 and/or 27 so that ubiquitination cannot occur together with an N-terminal domain exhibiting a destabilising N-terminal residue (e.g. arginine) and a replacement module comprising a modified PLB sequence such that it is unable to inhibit Ca2+-pump activity (e.g. N34A). It will be appreciated that the product in this embodiment indirectly affects degradation of the target moiety i.e. PLB by directing ubiqitination to the wild type which retains ubiquitination sites.

According to a further aspect of the invention there is provided an isolated nucleic acid comprising a modified or mutated form of a PLB nucleic acid sequence (vPLB) comprising at least one modification or mutation in the N-terminal domain and/or a deleted or mutated region that encodes a lysine residue in the expressed protein so that at least one lysine is not expressed in the protein encoded by the modified or mutated vPLB nucleic acid sequence.

Preferably, the deleted or mutated region of the vPLB affects lysine 3 residue and/or lysine 27 so that either or both of lysine 3 and 27 is/are not expressed in the protein encoded by the vPLB nucleic acid.

Preferably, the vPLB nucleic acid comprises both a modification in the N-terminal domain and a deleted or mutated region that encodes at least one lysine residue.

It will be appreciated that deletion or removal of the region encoding either or both lysine residue(s) in the expressed protein will render the vPLB protein resistant to ubiquitination since the modified/mutated vPLB protein no longer possesses a site for ubiquitin attachment and therefore a signal for degradation of the protein.

Preferably, the modification or mutation of the 5′ end of the nucleic acid polymer encoding PLB is such that the N-terminal domain of the protein comprises replacement of the region encoding a methionine residue at, preferably, position 1 with an unstructured linker sequence.

Preferably the linker sequence comprises a nucleic acid sequence encoding at least one glycine residue. More preferably still, the linker sequence further comprises an N-terminal residue of arginine or another destabilising residue. Studies have shown that inclusion of the N-terminal arginine residue on a target (model) protein confers a half-life of 2 minutes compared to 111 minutes of the wt PLB protein.

Preferably the linker sequence comprises a nucleic acid sequence encoding between 1-1000 residues and more preferably still a nucleic acid sequence encoding between 1-100 and more preferably still a nucleic acid sequence encoding between 1-50 and more preferably still a nucleic acid sequence encoding between 5-35 glycine residues. The minimum distance between the N-terminal residue and the lysine is 11 amino acids, although the efficiency of ubiquitination is reduced. Increasing the distance with an unstructured polyglycine stretch to 30-32 residues however, increases the efficiency of ubiquitination. It is thought that the important factor in determining the efficiency of ubiquitination is the segmental mobility of the region between the N-terminus and the target lysine. Accordingly, it will be appreciated that the number of glycine residues selected is intended to preserve segmental mobility without compromising the efficiency of ubiquitination and is not intended to limit the scope of the application. Residues other than glycine can be included in the linker sequence, but not lysine or any other acceptor for ubiquitin (or other degradation signal) attachment.

According to a further aspect of the invention there is provided a protein encoded by the vPLB nucleic acid of an aspect of the invention as hereinbefore described.

Accordingly it will be appreciated that the vPLB protein of the present invention orchestrates the destabilisation of wtPLB and its subsequent removal from the cell by proteasomal degradation.

In addition to reducing the steady state concentration of wild type PLB to zero or near zero, the vPLB replaces the wtPLB molecule. This provides the capacity to replace the target molecule with a designer molecule of our choosing. As such it will permit the retention of the beneficial features of PLB protein as well as the removal of those that maybe detrimental.

Preferably vPLB can be further engineered to contain any one or more of the mutations selected from the group comprising N34A-PLB (K_{Ca}=6.55), L31A E2A, L42A, I38A, L7A, F35A, I12A, R14A, V4A, I48A, R9A, L52A, P21A, V49A, R25A, Q26A, R13A, L28A, L39A, P21A and A24V+PLB (K_{Ca}=6.22).

The PLB mutations listed above show a range of inhibitory potential for the Ca²⁺-ATPase that may be useful in the design of the vPLB. The mutations listed above for PLB have K_{Ca} values ranging from those observed in the absence PLB (N34A) i.e. an inability to inhibit, to those in the presence of PLB (A24V).

It is desirable and advantageous to retain beneficial features, since proteins often perform multiple functions. For example PLB inhibits SERCA2, but also appears to function as an ion channel¹⁵. Deletion of some functions but not all may prove to be of benefit in the management of human disease. For example, deletion of inhibitory influence on SERCA2, but not ion channel activity might be therapeutic. In the present invention we provide molecules for the novel strategy capable of removing native or wtPLB from a biological system and replacing it with a ‘designer’ vPLB or mutant form of the target protein, which retains the beneficial qualities (for good health) but lacks the qualities which contribute to disease, and the qualities recognised by the selective degradation domains of the chimera. In this way the vPLB can be exported to other contexts, where removal and replacement of the target molecule is a superior strategy to elimination of the target molecule. In the present invention we have sought to manipulate the degradation of PLB protein as a strategy to control the steady state level of the protein. Mindful of the adverse effects of PLB protein ablation in man⁵, the strategy of the present invention allows for the advantageous elimination of natural/native PLB protein but replaces it with a modified or mutant PLB molecule (vPLB protein) according to the design of the present invention.

vPLB protein provides a new flexibility to eliminate qualities detrimental to the organism whilst retaining others found to be beneficial. The approach exploits the oligomeric nature of PLB and employs trans-ubiquitination⁶ to mark wild type PLB protein for degradation by the proteosome. We believe that vPLB protein directs the marking of wtPLB forms for degradation through the process of trans-ubiquitination. vPLB protein encodes a signal for ubiquitination in an extended N-terminal domain, but does not possess an amino acid receptive to ubiquitination by virtue of the manipulation/engineering/design of the present invention. Association of vPLB protein with wtPLB protein facilitates the ubiquitination and rapid destruction of wtPLB protein driven by molecular cues engineered into the vPLB nucleic acid molecule encoding the vPLB protein or the vPLB protein itself. Demonstration of this is presented hereinafter in a model eukaryotic system.

According to a yet further aspect of the invention there is provided a vector or delivery vehicle comprising the nucleic acid or protein encoded thereby of the present invention.

As used herein, the term “vector” is used in reference to nucleic acid molecules that transfer DNA segment(s) from one cell to another. Vectors are used to introduce foreign DNA, in this instance vPLB nucleic acid into host cells where it can be replicated (i.e., reproduced) in large quantities. The term “vehicle” is sometimes used interchangeably with “vector.” Vectors, including “cloning vectors” allow the insertion of DNA fragments without the loss of the vector's capacity for self-replication. Cloning vectors may be derived from viruses, plasmids or genetic elements from eucaryotic and/or procaryotic organisms; vectors frequently comprise DNA segments from several sources. Examples of cloning vectors include plasmids, cosmids, lambda phage vectors, P1 vectors, yeast artificial chromosomes (YACs), and bacterial artificial chromosomes (BACs).

Preferably, the vector may also comprise a promoter for driving expression of the nucleic acid of the present invention.

According to a yet further aspect of the invention there is provided a host cell transformed with the vector comprising the nucleic acid of the present invention.

According to a further aspect of the invention there is provided a use of vPLB nucleic acid or the protein encoded thereby or the protein itself as hereinbefore described as a pharmaceutical.

Preferably, the vPLB nucleic acid or protein encoded thereby or the vPLB protein itself is formulated as a pharmaceutical composition in an appropriate carrier, diluent or excipient.

According to a yet further aspect of the invention there is provided a method of producing vPLB nucleic acid molecule comprising:
(i) modifying or mutating native or natural PLB so that a region encoding of a methionine residue at the N-terminus of the protein is replaced with an linker sequence comprising a sequence of nucleic acids encoding at least one glycine residue and at least one arginine residue or other destabilising residue at the N-terminus of the vPLB protein; and
(ii) deleting or mutating a region of nucleic acids that encode at least one or both lysine residue(s) from the original PLB sequence so that lysine(s) is/are not expressed in the protein at a location to participate in N-end rule directed ubiquitination.

It will be appreciated that the method may be performed with step (ii) preceding step (i), the order of the sequence is not essential to the execution of the invention, so long as the resultant vPLB nucleic acid contains the modifications/mutations which dictate the limitations as hereinbefore described.

Preferably, the method further includes the step of further modifying the vPLB as hereinbefore described so as to retain beneficial qualities and to eliminate those considered detrimental.

Preferably, the method includes any one or more of the features as hereinbefore described.

According to a further aspect of the invention the method further includes the expression of the vPLB protein from the nucleic acid of the present invention.

According to a yet further aspect of the invention there is provided a method of treating cardiac disorders selected from the group comprising acute congestive heart failure precipitated by mycardial ischemia, hypertrophic cardiomyopathy, dilated cardiomyopathy, which have a common feature of diastolic dysfunction and lethargic Ca²⁺handling by the SR comprising administering a therapeutically effective amount of vPLB nucleic acid or a vector comprising such nucleic acid or a protein expressed by such nucleic acid, to a subject in need of treatment for the specified conditions.

The vPLB nucleic acid of the present invention or protein encoded thereby or the vPLB protein itself of the present invention may be administered by oral, intra-venous, intra-cardiac, intra-muscular or any other route deemed appropriate by a physician. vPLB nucleic acid or protein may be administered as a pharmaceutical composition/formulation including when it is incorporated in a vector or delivery vehicle.

According to a yet further aspect of the invention there is provided use of primers as set forth in SEQ ID NOS; 2, 3, 4, 5, 6, 7, 8, 9, 10 or 11 in the method of the present invention.

The invention will now be described by way of example only with reference to the following Figures wherein:

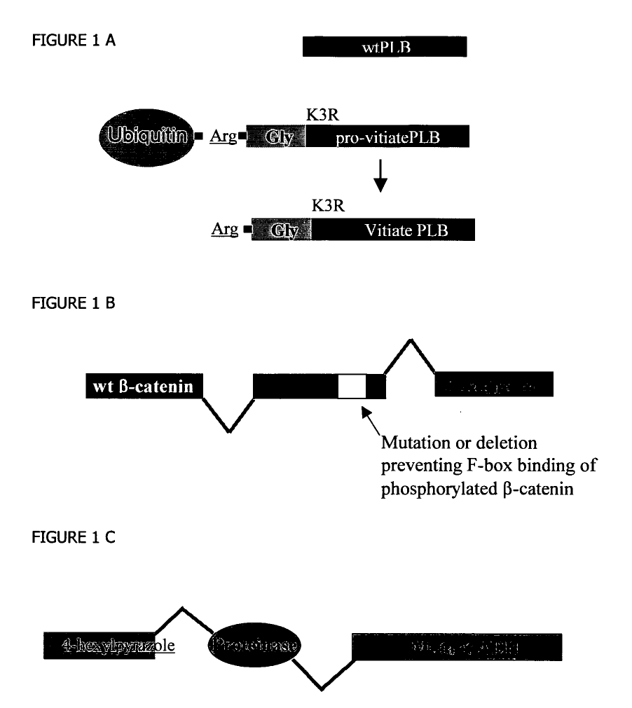

FIG. 1 A illustrates the schematic describing the design features of vPLB; the vPLB gene represents a fusion protein of PLB and ubiquitin connected by a short glycine linker region with an arginine at the N-terminal end of the linker. The deubiquitinating enzymes cleave after the final glycine of ubiquitin. Once synthesised the fusion protein (pro-vitiate PLB) is deubiquitinated by endogenous ubiquitin specific proteases (ubiquitin is cleaved after its C-terminal glycine) to yield a modified form of PLB (vPLB) with a destabilising N-terminal arginine residue.

FIG. 1B(i) illustrates application of applying the method of the present invention to engineered F-box proteins showing a schematic description of targeting, destruction and replacement motifs,

FIG. 1B(ii) Sequence of a potential β-catenin molecule (Seq ID No 12) which is schematically represented in FIG. 1B(i). Mutations or deletions have not been incorporated, and therefore the sequence between the linkers (in black) represents an intact β-TrcP domain).

FIG. 1C illustrates the application of the method of the present invention to catalytic antagonists. The targeting moiety is an inhibitor of ADH that brings the proteinase domain into close proximity of the target ADH. The replacement domain is a functional mutant ADH molecule that is insensitive to both proteolytic cleavage and inhibition by the targeting motif.

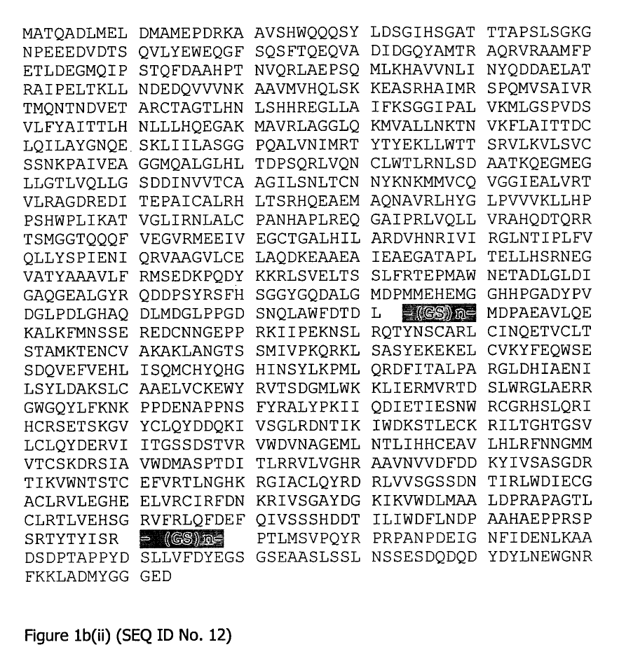

FIG. 2 shows the proposed mechanism of vPLB action; the formation of mixed oligomers of vitiate and wild type PLB would result in trans ubiquitination of wild type PLB and its subsequent removal from the ER and degradation by the proteasome.

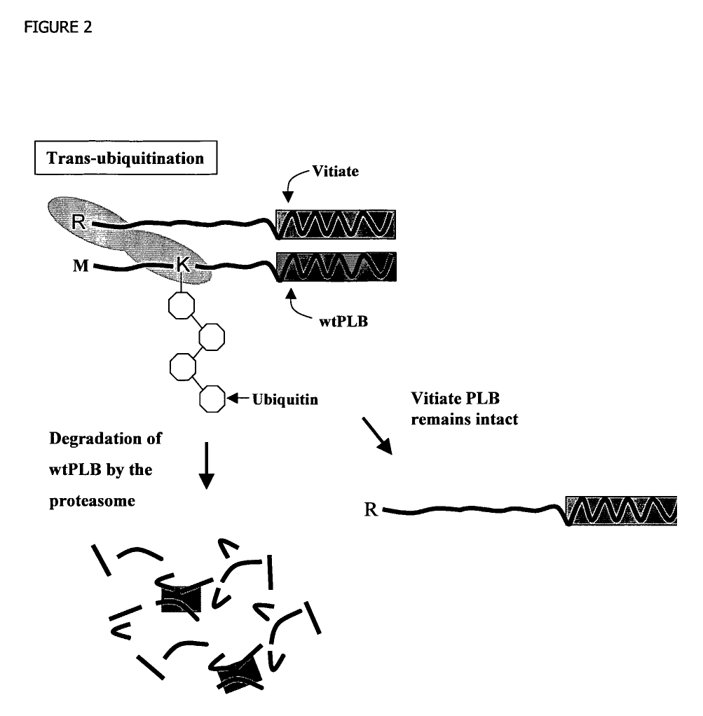

FIG. 3 illustrates the heterologous expression of wild type PLB in S. cerevisiae; the PLB gene was subcloned into the yeast shuttle vector pEMBLyex and the vector subsequently transformed into the wild type strain of S. cerevisiae BSL1 11B. Positive transformants were selected on a URA3 dropout media. Cells were then grown on a SDgal/raf media and aliquots of cells harvested at various time points. Cell lysates were analysed by immunoblotting using A1 antibody. Panel (A) Time course of PLB expression. Panel B, Densitometric quantification of PLB levels. Panel C PLB levels expressed as a ratio of densitometric value to OD₆₀₀ of culture sampled.

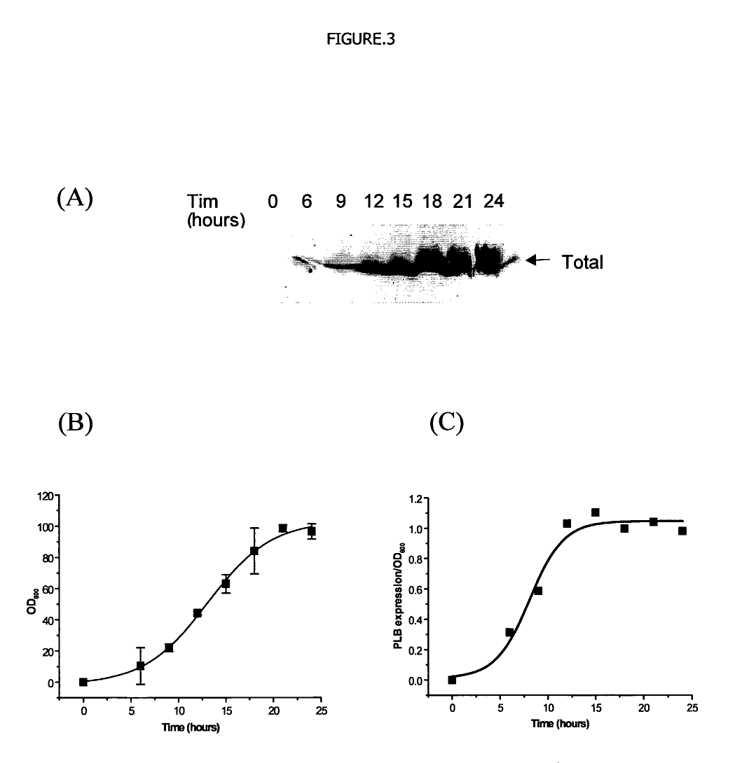

FIG. 4 shows that the heterologously expressed PLB is targeted to the endoplasmic reticulum of S. cerevisiae; cells were grown on SDgal/raf media to mid log phase and harvested. Cells were then spheroplasted and lysed prior to loading onto a sucrose density step gradient. The resulting gradient was fractionated and analysed by immunoblotting using A1 antibody. Spheroplasted cells were also processed for immunofluoresence microscopy (see materials and methods). Panel A(i) Schematic of the sucrose gradient. A(ii) Protein concentration across fractionated gradient. A(iii) Immunoblot analysis for PLB (A1 antibody) and the ER marker dpm1p (anti-dpmlp antibody). Panel B, Cells dual labelled with concanavalin A (panels ii, v, vii) and either A1 (panels i, iii, for cells expressing wtPLB and panels iv, vi, for cells expressing vPLB) or α-Vma2p for the vacuole (panels vii, ix). Individual labeling patterns are displayed in left and central columns of figure, the overlay of individual labeling patterns is displayed in right hand column of the figure. All cells were also stained with DAPI. Arrows indicate perinuclear staining [P] and sub-plasma membrane staining [SP].

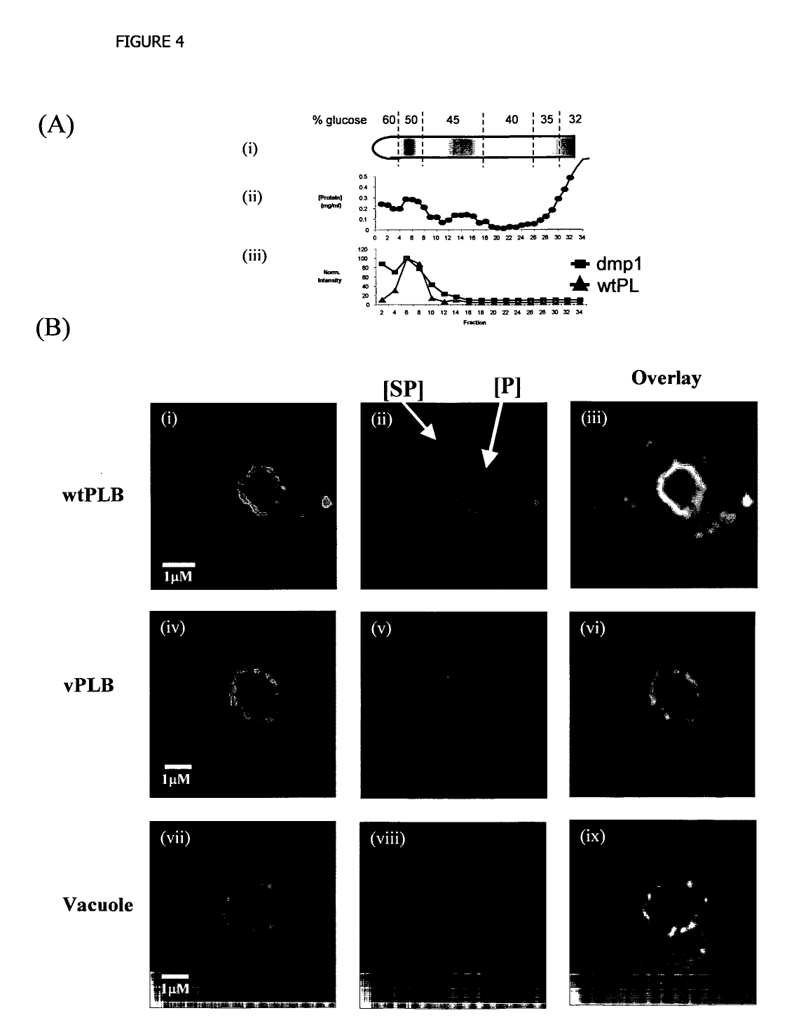

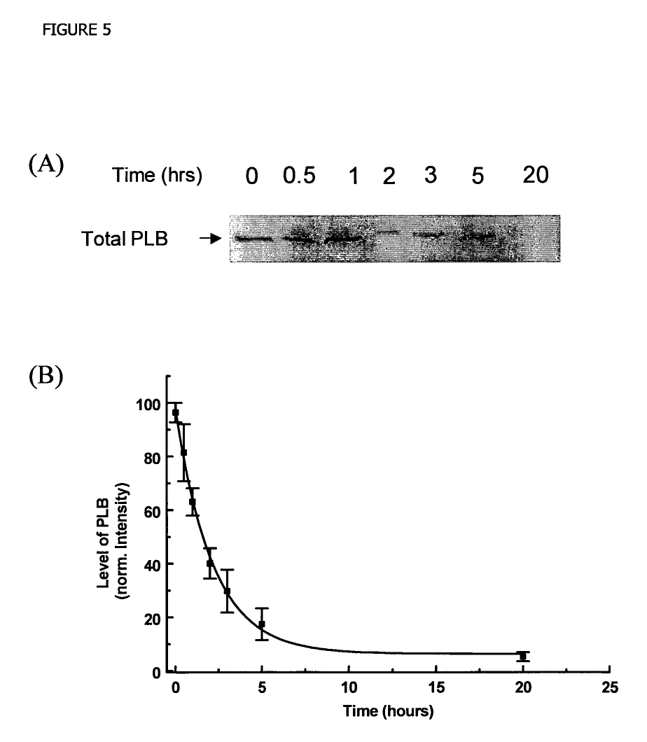

FIG. 5 shows the turnover of heterologously expressed wild type PLB in S. cerevisiae; expression of PLB protein was achieved by overnight growth in SDGal/raf media. Expression was then terminated by resuspending cells, after thorough washing, in SDGlu media. Aliquots of cells were removed at various time points thereafter. Resulting cell lysates were analysed for residual PLB by immunoblotting using anti-PLB antibody A1. (a) Immunoblot showing the removal of PLB over time. (b) Immunoblot data was quantified using densitometry. A monoexponential decay was fitted and a half time of 111+/−7.5 minutes calculated. FIG. 6 shows vPLB can be separated from wild type PLB by standard tricine PAGE; the individual clones pYES2:pro-vitiate PLB and pMAH3:wtPLB were grown in SDgal/raf media to mid log phase. Cells were harvested and total cell lysates prepared. Proteins were separated on 16% tricine gels, transferred to PDVF membrane and immunoblotted using A1 antibody. (A) Lane (i), Vitiate PLB. (ii) Wild type PLB. (iii) A mixture of vitiate and wt PLB.

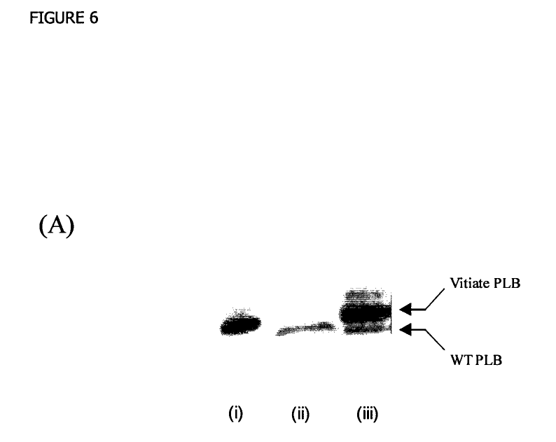

FIG. 7 shows vPLB accelerates the catalytic removal of wtPLB from the S. cerevisiae; cells were grown to early-mid log phase in SDglu media (steady state wtPLB expression), harvested, washed and resuspended in SDgal/raf media (induction of vitiate PLB expression). Cells were removed at various points thereafter and the levels of both wtPLB and vitiate PLB determined by immunoblotting. Panel A(i), expression levels of wtPLB (lower band) and vitiate PLB (upper band) in SDgal/raf media over a 21 hour period. Panel A(ii), steady state levels of wtPLB in cells containing a ‘naked’ pYES2 vector as well as pMAH3 (constitutive expression of wt PLB) over a 21 hour period in SDgal/raf media. Panel B represents the quantification of immunoblot data, (), wtPLB expression levels in SDgal/raf, (▪) vitiate PLB expression levels in SDgal/raf and (♦), wtPLB expression levels in SDgal/raf in the absence of vitiate PLB expression.

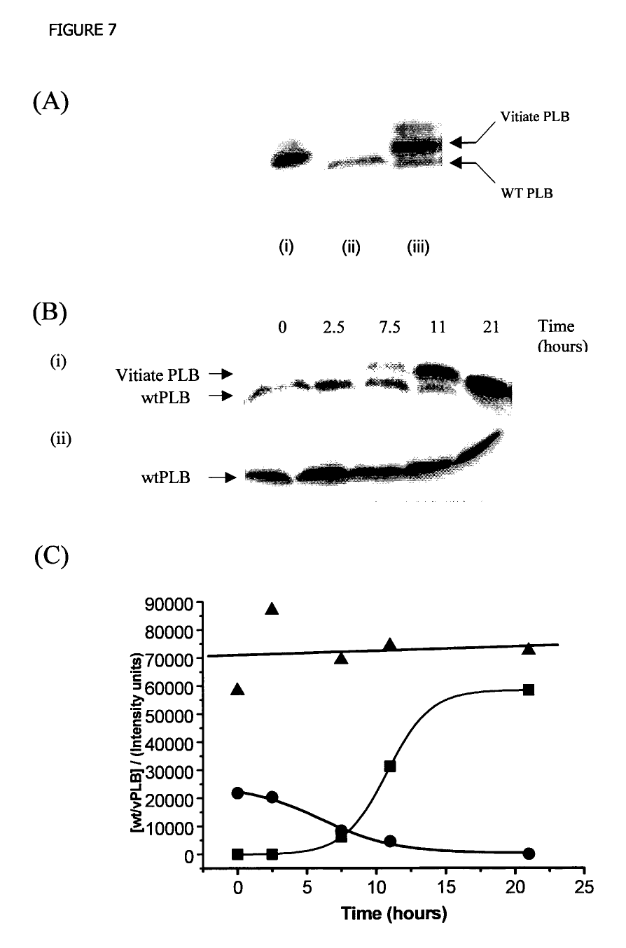

FIG. 8 shows the co-expression of Vitiate PLB reduces the steady state level of wtPLB. The A1 epitope of vitiate PLB was removed and a c-Myc epitope tag engineered onto the c-terminus of the molecule to allow detection. Both molecules were co-expressed in S. cerevisiae with wtPLB under the control of a constitutive promoter (i.e. constantly active), whereas vitiate PLB-cmyc was placed under the control of a switchable promoter. Once cells had reached steady state wtPLB expression, vitiate PLB-cmyc expression was turned on. Samples were taken at time zero and after 8 hours of co-expression.

The left panel of FIG. 8 shows a western blot probed with the a-PLN antibody A1. At time zero a robust level of wtPLB expression is observed, however, by 8 hours of co-expression the level wtPLB is undetectable. The right panel of FIG. 8 shows the same samples probed with a a-c-Myc antibody in order to detect vitiatePLB-cmyc. As expected at time zero vitiatePLB-cmyc is undetectable, but by 8 hours a robust steady state expression is observed

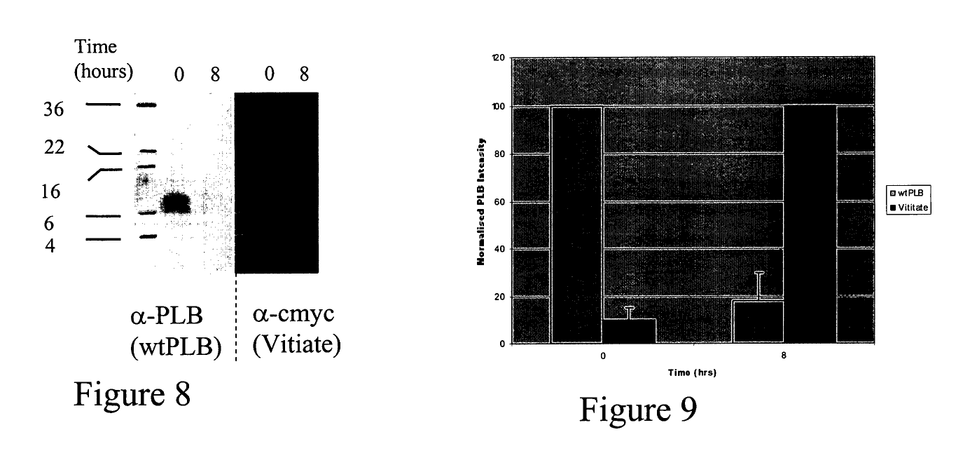

FIG. 9 shows densiometric quantification of the data shown in FIG. 8.

### DETAILED DESCRIPTION OF THE INVENTION

### Materials and Methods

Heterologous expression of PLB: Heterologous expression of PLB in S. cerevisiae was achieved by inserting the PLB gene into the yeast shuttle vector pEMBLyex. The vector pEMBLyex provides the GAL1 promoter for inducible expression of heterologous proteins, as well as a gene for uracil biosynthesis that serves as an auxotrophic marker in yeast, and ampicillin resistance for selection in E. coli. Thus, subcloning was achieved by altering both the N and C-terminal restriction sites of the PLB gene from NdeI and XhoI to BamHI and HindIII respectively, in order to make them compatible with the multicloning site of pEMBLyex. The mutagenic primers 5′-CGGGATCCATGGATAAAGTCCATACC-3′ (SEQ ID NO:2) and 5′-CCCAAGCTTTTAGAGAAGCATCAAATG-3′ (SEQ ID NO: 3) were used in the PCR together with the vector pet28a:PLB as a template. The resulting BamHI, HindIII fragment was purified, digested (with BamHI and HindIII) and ligated into the multicloning site of pEMBLyex. The product of ligation was subsequently transformed into supercompetent XL-Blue E. Coli cells. Positive transformants were selected by ampicilin resistance and screened by colony PCR using PLB gene specific primers MSBPLB(F), 5′-GCGGGATCCATGGATAAAGTCCATACC (SEQ ID NO:4) and MSBPLB(R), 5′-CCCAAGCTTTTAGAGAAGCATCAAATG-3′. (SEQ ID NO:5) Positive clones were then confirmed by sequencing.

Constitutive expression—The vector pMAH3 contains a single BamHI restriction site separating the PMA1 promoter from the terminator region. The PMA1 promoter drives constitutive expression of heterologous proteins. Therefore, in order to subclone PLB into this site the 3′-HindIII restriction site of the PLB in pEMBLyex was changed to a BamHI site using the mutagenic primer 1PLBHTOB(R), 5′-CCTTTGATATTGGATCCTAAGCTTTTAGAG-3′ (SEQ ID NO:6) together with MSBPLB(F) in a standard PCR. The PCR product was purified, ligated into pMAH3 and transformed into supercompetent XL-Blue E. Coli cells. Positive transformants were selected by ampicilin resistance and screened by colony PCR using PLB gene specific primers MSBPLB(F) and MSBPLB(R). Positive clones were also confirmed by sequencing.

Yeast transformation: Yeast BSL1-11B wild type cells were transformed using the standard lithium acetate method⁷. Competent cells were prepared by growing cells to mid log phase, these were then harvested by centrifugation at 1000 g (5 minutes), washed with sterile H₂O and resuspended in 100 mM lithium acetate, 10 mM Tris-HCl (pH 7.5), 1 mM EDTA (LiAc/TE buffer). Competent cells (100 μl) were added to the transformation mix, which also included plasmid DNA (1 μg), carrier DNA (salmon sperm DNA, 160 μg), 32% PEG (Mw: 3350)/ LiAC/TE and 0.01% v/v DMSO. The transformation mix was vortexed and then incubated at 30° C. for 30 minutes and heat shocked at 42° C. for 15 minutes. Cells were then harvested, resuspended in sterile H₂O and plated onto minimal glucose media (SDglu) minus uracil (auxotrophic selection) and incubated at 30° C.

Total protein lysates from yeast cells were prepared by first removing the cell wall and then incubating in lysis buffer. Thus cells were harvested by centrifugation at 1000 g, washed once in sterile H₂O and resuspended in phosphate buffered saline (pH 7.2), 1.2M Sorbitol, 10 mg/ml yeast lytic enzyme (ICN), 1 mM DTT. Cells were incubated at 30° C. for 90 minutes. The resulting spheroplasts were harvested by centrifugation at 1000 g for 5 minutes and washed once in sterile H₂O, before being resuspended in lysis buffer, (PBS (pH 7.2), 0.5% SDS, 1% protease inhibitor cocktail (Sigma)). Lysis was allowed to continue for 45 minutes at RT, prior to harvesting solubilised proteins by centrifugation at 11000 g and collecting the supernatant. Proteins from the supernatant were precipitated by mixing one part supernatant with 9 parts ethanol/acetone (50:50) and incubated on ice for 10 minutes. Precipitated proteins were recovered by centrifugation, air dried and resuspended in PBS, 0.1% SDS. Protein concentrations of total protein lysates were measured using the standard bicinchoninic acid method⁸.

Immunoblot analysis: Total yeast proteins were separated by both SDS (15%) and tricine (16%) polyacrylamide gel electrophoresis (PAGE). Once separated, proteins were electro-transferred to PDVF membrane (Pall BioSupport, Portsmouth, UK) by wet blotting and non-specific binding sites blocked for 1 hour at room temperature using 5% dried milk in 50 mM Tris buffered saline, (pH 7.4), 0.1% Tween 20. Membranes were then probed overnight with anti-PLB antibody (A1; 1:5000) at 4° C. Horseradish peroxidase conjugated goat anti mouse secondary antibody together with_an enhanced chemiluminesent detection system (Supersignal West Pico Chemiluminesent, Pierce) was used to visualise primary antibody. Data were captured using a Fuji Las-1000 Imaging System with a CCD camera (connected to a Pentium II PC; including AIDA software for analysis).

Subcellular Fractionation: Wild type BSL1-11B cells transformed with pEMBLyex:wtPLB were grown to mid log phase in SDgal/raf expressing media. Cells were then harvested by centrifugation at 1000 g for 5 minutes and resuspended in buffer A (0.1M potassium phosphate buffer (pH 7.5), 1.2M Sorbitol) with 0.2% □-mercaptoethanol, 10 mg/ml Zymolase 20T (approximately 5 mg/g cells) and incubated for 35 minutes at 37° C. The resulting spheroplasts were washed with 0.6M Sorbitol, 25 mM MES-KOH, pH 6, 1 mM PMSF, subsequently resuspended in the same buffer and homogenised using ten strokes in a dounce homogeniser. Membranes were then layered onto a discontinuous sucrose density gradient consisting of 60%, 50%, 45%, 40%, 35% and 32% (w/v) sucrose steps in 10 mM MES-KOH, pH 6, 1 mM KCl, 1 mM EDTA, 0.1% v/v ethanol and 1 mM MgCl₂ and centrifuged at 141000 g for 16 hours at 4° C. Gradients were then fractionated starting from the bottom of the tube and the protein concentration of each fraction determined using the standard BCA assay. Fractions were then analysed by SDS-PAGE and immunoblotting using the A1 antibody⁹.

Immunofluoresence: Actively growing cells at mid log phase were fixed by the addition of paraformaldehyde (5% v/v) to the culture media and incubated for 1 hour at room temperature. Cells were centrifuged at 700 g and washed twice with buffer A (0.1M potassium phosphate buffer (pH 7.5), 1.2M Sorbitol). Cells were subsequently resuspended in Zymolase 20T cell wall digestion mix (buffer A, 0.2% β-mercaptoethanol, 10 mg/ml Zymolase 20 T (approximately 5 mg/g cells) and incubated for 35 minutes at 37° C. The resulting spheroplasts were pipetted (20 μl) onto a microscope slide and allowed to attach for 10 minutes. Excess cells were removed by aspiration and attached cells permeabilised by the addition of SDS (0.5% in PBS) for 10 minutes. The SDS was removed and the permeabilised cells washed 10 times with filtered sterile H₂O. Taking care not to let cells dry out, primary antibodies (A1 staining for PLB and α-Vma2p staining for subunit B of the vacuolar proton ATPase) at a dilution of 1:200 were then placed onto cells and incubated for 2 hours at room temperature. Rhodamine conjugated conconavalin A was used as an ER marker at a dilution of 1:200. Primary antibody was then removed, cells washed 10 times with sterile H₂O and a mixture of FITC conjugated goat anti mouse secondary antibody (1:100) and DAPI (1:500) applied. After a 1 hour incubation at room temperature cells were washed 10 times with sterile H₂O and mounted using Vectamount (5 μl). Wide field images of fluorescent samples were acquired using a 100× (pixel size of 0.06 mm), 1.4 NA oil immersion objective lens on a DeltaVision restoration system (Applied Precision Inc., Issaquah, Wash.), based around an Olympus IX-70 inverted microscope equipped with 100W mercury-arc illumination, UV, fluorescein and rhodamine excitation and emission filter sets. Optical sections 0.15-0.2 mm apart were captured with a CoolSNAP HQ CCD camera (Roper Scientific, Tucson, Ariz.). Digital deconvolution and image analysis was then performed on the data sets with SoftWoRx deconvolution software (Applied Precision Inc.)

Measuring the rate of degradation: Turnover of heterologously expressed PLB was measured by analysing the removal of PLB from cells once synthesis was terminated. Expression of PLB, in BSL1-11B cells transformed with pEMBLyex:PLB, was switched on by growing in minimal galactose media (SDgal); steady state PLB expression was achieved by growing cells to mid log phase. Synthesis of PLB was subsequently terminated by harvesting cells (centrifugation at 1000 g for 5 minutes), washing twice and re-suspending in SDglu media. Cells were allowed to continue growth and aliquots were removed at various times thereafter. Cell lysates were prepared and analysed by gel electrophoresis and western blotting using the A1 antibody.

Construction of vitiate PLB: The vitiate molecule is a fusion protein composed of three moieties—Ubiquitin linked to PLB by a short glycine linker region. The vitiate construct was synthesised by performing two separate PCR reactions. In the first reaction the PLB gene was used as a template together with the reverse primer (MSBPLB(R)) and a forward primer (PLBEXT(F)) incorporating a substantial 5′ extension 5′-CGGTGGGGGAGGCGGTGGGGGAGGCGGATCCATGGATAGA GTCCA-3′ (SEQ ID NO:7). The extension formed the basis of the linker region and was therefore designed to encode for glycine residues. A second PCR was performed using the ubiquitin gene as a template with a gene specific forward primer, UBXB1(F); 5′-CATCTCTAGAACCTGCAGGGAATGCAGATCT TCGTG-3′ (SEQ ID NO:8), and a reverse primer (UBEXT(R)), 5′-CCCCACCGCCTCCC CCACCGCCTCCCTCGAGACGGCCGCCCCTCA-3′ (SEQ ID NO:9), with a 3′ extension designed to incorporate complementation to the PLBEXT(F) primer. The PCR products from these two separate PCRs were used in a third PCR as templates together with gene specific forward and reverse primers for ubiquitin and PLB respectively. The resulting PCR product was then ligated into pEMBLyex and transformed into E. Coli (XL-Blue supercompetent cells). Positive transformants were screened by colony PCR using UBXB1(F) and MSBPLB(R) primers, and subsequently confirmed by sequencing. The vitiate PLB gene was also subcloned into the vector pYES2. In order to make compatible sticky ends for the vector the 5-′ and -3′ restriction sites of vitiate PLB, Xba1 and HindIII, were reversed . This was achieved using the mutagenic primers XBTOHIND(F), 5′-GGGCCGCTCTAAAAC CCGCAGGGA-3′ (SEQ ID NO:10) and HINDTOXB(R), 5′-AAGCCTCTAGAGA A GCATCACAAT-3′ (SEQ ID NO:11), in a standard PCR reaction using pEMBLyex:vitiate PLB as a template. pYES2:vitiate PLB was transformed into the wild type strain of S. cerevisiae using the standard lithium acetate method.

Testing the effectiveness of vitiate PLB: Wild type BSL1-11B cells were co-transformed with pMAH3:wtPLB and pYES2:vitiate PLB. Positive double transformants were selected on SDglu minus uracil and leucine dropout media. In order to examine the efficacy of vitiate PLB, double transformants were grown in SDglu media to early/mid log phase (OD₆₀₀˜1.0). Cells were harvested, washed twice and resuspended in SDgal media (inducing vitiate expression) to an equivalent OD₆₀₀ and allowed to continue growth. Samples were removed at this point (denoted time zero) and at various times thereafter. The presence of both wtPLB and vitiate PLB was detected by tricine PAGE and immunoblotting as described above.

### EXAMPLE 1

A protein “knockdown” strategy has been proposed by Cong et al¹⁰, where a chimeric protein consisting of β-TrcP (which is an example of an F-box protein) fused to the β-catenin binding domain of E-cadherin (amino acids 794-883) is produced. E-cadherin binds the non-phosphorylated form of β-catenin i.e. the stabilised form that is observed in some human cancers. The strategy therefore combines both targeting and destruction (F-box recruitment to the SCF E3 ligase) motifs. However, using the method of the present invention we are able to improve on the utility and efficiency of the prior art by providing a product which also advantageously includes a replacement motif. This may be achieved by making two important changes to the chimeric molecule described by Cong et al. Firstly, introduction of a replacement wild type β-catenin domain (i.e. phosphorylation sites intact). Secondly, removal of the features of the F-box domain that allow it to bind the phosphorylated form of β-catenin (FIG. 1B) by either deletion of the protein-protein interaction domains (i.e. the WD40 repeats/leucine rich repeats) or by mutating those residues within these domains involved in binding (refer to SEQ ID NO:12). Thus, the F-box protein fused to the β-catenin binding domain, E-cadherin is able to remove the mutant (cancerous) β-catenin molecules, whilst the wild type β-catenin replacement domain restores normal wnt/β-catenin signalling without any intramolecular interactions with the F-box domain of the construct.

### EXAMPLE 2

Utilising the method and product of the invention, horse liver alcohol dehydrogenase (HLADH) was the target protein and a potent inhibitor is used as the targeting ligand (4-hexylpyrazole; K_{I}=10⁻⁸M). The targeting ligand can be chemically linked to the subtilisin proteinase to make the CA. In a mixture of proteins the CA is able to increase HLADH degradation by subtilisin by 29.3 fold over free subtilisin. Human ADH may contain ‘loss of function’ mutations that manifests as a human metabolic disease. Addition of a normal wild type human LADH domain to the CA described would result in a molecule with not only targeting and destruction motifs, but also a replacement motif (FIG. 1C). This tri-partite molecule would therefore target the non-functional mutant protein, destroy it and replace it with a functional protein restoring normal metabolic activity. Importantly, the substrate recognition motif of the proteinase employed would have to be engineered out of the replacement domain in order to prevent any potential auto-proteolysis. Also, the replacement molecule would have to be altered in such a way to substantially reduce its sensitivity to the targeting motif, which is an inhibitor of ADH, thus maintaining function.

### EXAMPLE 3

Generically, the vitiate principle incorporates three distinct functional modules—A targeting domain (module 1) fused to a destruction domain (module 2) and a replacement domain (module 3). Targeting is generally elicited by binding. Binding partners may include antibody specific for target protein X, specific peptides or aptomers or drug like chemical entities. The destruction domain (module 2) either directly degrades the target or initiates destruction of the target by activating the endogenous degradation machinery. The replacement domain (module 3), differs from the original sequence of the target protein X by at least one residue and is not substantially inactivated by either modules 1 and 2.

### EXAMPLE 4

vPLB protein is a modified form of PLB protein which has been designed to stimulate the catalytic removal of wild type PLB protein from the cell. It is able to achieve this effect because of two important features (FIG. 1A). Firstly, vPLB protein has a modified N-terminal domain in which the normal methionine at residue 1 has been replaced by an unstructured linker sequence comprising for example Arg-Leu-Glu-(Gly)₇-Ser which provides the protein with a new N-terminal residue, arginine. According to the N-end rule¹¹, arginine is a destabilising residue and therefore confers a short half-life (2 minutes) upon a protein. Instability is manifest by the covalent attachment of ubiquitin to lysine residue (s) situated ideally 15-17 residues from the N-terminus¹¹. This targets the protein for destruction by the proteasome. vPLB protein has been engineered to lack lysine residues through the replacement of lysine 3 and/or 27 (in the original PLB nucleic acid sequence of some species) with a region encoding arginine. Thus, although vPLB protein has a destabilising N-terminal residue, the absence of a lysine residue renders it stable as it does not possess a site for ubiquitin attachment (and therefore signal degradation). The heterologous expression of vPLB protein bearing an arginine at the N-terminus was achieved by expressing an ubiquitin-vPLB fusion protein. Post-translational cleavage of ubiquitin occurs liberating vPLB as one product (FIG. 2) and ubiquitin as second. A fusion protein other than the ubiquitin-vPLB construct would suffice where the alternative protein (module 0)-vPLB construct was subject to post-translational cleavage events in a controlled, site specific manner between module 0 and vPLB. There are a number of examples of this type post translational processing apart from the ubiquitin fusion protein described above. For example, some viruses express there genes as long polyproteins. The polyprotein is then processed by specific proteases into a number of smaller, mature, functional proteins¹⁷. A second example is the mechanism of concanavalin A processing. Here, the initial product of translation is first deglycosylated and cleaved into smaller polypeptides before being rearranged and annealed to form the mature protein. Thus the N-terminal residue of the initially translated protein has been changed¹⁶. As such, the formation of mixed oligomers of vitiate and wild type PLB could result in trans ubiquitination of wild type PLB and its subsequent removal from the ER and degradation by the proteasome as depicted in FIG. 2.

### EXAMPLE 5

This present invention provides a means to control the steady state level of expression of a protein (e.g. wild type PLB) by co-expression of a corrupting partner (vPLB) and to replace the original protein with an alternative form of the protein. The corrupting partner binds to the target protein and stimulates the degradation of the target protein. The corrupting partner contains a module to replace the target protein, this replacement module contains some, but not necessarily all of the features of the target protein. The replacement module differs from the target module in at least one respect.

Heterologous Expression of PLB in S. cerevisiae: Sacchromyces cerevisiae was used as the model system for studying PLB expression and degradation in this study. It represents a fast growing, eukaryotic cell, which offers a diverse range of mutant strains altered in their protein degradation capability. As PLB has not been expressed in yeast previously, we optimised conditions for the expression of significant levels of PLB and then examined the sub-cellular location of PLB. In this section of the study, PLB expression was driven by a galactose dependent promoter (GAL1 in the vector pEMBLyex) and the immunodetection of PLB was evident following the transfer of cells to a galactose/raffinose based growth medium (FIG. 3A).

The amount of PLB in total cell lysates increased with time to a peak seen following 20 hours of expression (FIG. 3B). PLB expression per cell however, reached steady state earlier than this, at 12 hours (FIG. 3C), after which point the increase in total PLB was the result of an increase in yeast cell number alone. All subsequent experiments were performed with yeast in the logarithmic growth phase, at which point maximal levels of expression of wild type PLB were achieved.

### EXAMPLE 6

Targeting of heterologously expressed PLB: Heterologous expression of proteins can proceed to an abnormally high level, at which point inappropriate localisation of the protein can occur. PLB is a small membrane protein found in the SR of cardiac myocytes, the corresponding organelle in S. cerevisiae is the ER, and thus a series of experiments were performed to determine the subcellular location of PLB at steady state expression (12 hours post induction). In the first of these studies, membranes were prepared from yeast and these were fractionated on a discontinuous sucrose gradient (modified version of the protocol as described by Song et al¹²). Three distinct fractions were resolved as represented schematically in FIG. 4A. The identification of endoplasmic reticulum derived fractions was established by immunodetection of the endogenous ER protein dolichol phosphate mannose synthase (dpmlp, FIG. 4A(iii)). This ER marker was enriched in fractions 2-12, which represent the most viscous section of the gradient. Phospholamban, detected with monoclonal antibody A1⁹, was found in the same fractions as dmplp, confirming the ER localisation of PLB. Heterologously expressed PLB protein co-fractionates with the ER marker indicating that PLB, as anticipated, does target to the yeast endoplasmic reticulum.

In order to corroborate ER localisation, S. cerevisiae cells expressing wtPLB (or vPLB) at steady state (grown to mid log phase) were fixed in paraformaldehyde. Spheroplasts were prepared; these were permeabilised with SDS (0.1% w/v) and stained with conconavalin A (ConA). Con A is a lectin that is able to bind sugar residues found specifically on the proteins of ER/Golgi and is therefore a commonly used ER/Golgi marker²⁷. Deconvolution confocal microscopy was used to visualise conA staining (FIG. 3B(ii, v, viii)), cells stained with ConA display a ring of perinuclear staining with spoke like elements radiating out to the extremities of the cell. Punctate sub plasma membrane staining was also observed. This staining pattern agrees well with the current understanding of the structure of the ER in S. cerevisiae³³. The same cells stained for wtPLB (A1 antibody and visualised by using a FITC labeled secondary antibody) displayed a robust perinuclear labelling pattern (FIG. 3B(i)), coincident with part of the ER/Golgi system defined by concanavalin A staining. Furthermore, superimposition of these two staining patterns (i.e. cells dual labelled with A1 (wtPLB) and conA (ER marker)) showed extensive overlap between staining pattern (yellow perinuclear and sub-plasma membrane staining) confirming localisation of wtPLB in the ER. Vitiate PLB also described the same perinuclear pattern of staining (FIG. 3B(iv), which again co-localised with conA (FIG. 3B(vi) indicating that mutations introduced into wtPLB in order to make vPLB do not affect targeting. Finally, an anti-P₀ H⁺-ATPase antibody (α-Vma2p) was used to visualise the vacuolar membrane in S. cerevisiae cells expressing wtPLB. The staining pattern described a large membrane bound compartment (FIG. 3B(vii)), spatially distinct from the perinuclear pattern observed for the ER marker ConA (FIG. 3B(viii) and PLB (FIG. 3B(i)). This is more clearly demonstrated when these images are superimposed (FIG. 3B(ix)). Thus, both wtPLB and vPLB appear to reside in the endoplasmic reticular membrane and are not targeted to the vacuole.

### EXAMPLE 7

Turnover of heterologously expressed PLB: In advance of the examination of the effects of vitiate PLB on the steady state expression of PLB, we examined the turnover of wild type PLB expressed in yeast. The heterologous expression system described here provides a convenient method of measuring protein turnover. We can measure the turnover of heterologously expressed PLB by simply terminating synthesis and measuring the level of PLB remaining over a period of time (analogous to conventional chase period of a pulse chase experiment). Cells were grown to steady state PLB expression (mid log phase) in SDgal medium, at which point cells were harvested, washed twice and resuspended in a non-expressing media (SDglu; glucose is a potent repressor of the Gall promoter) and allowed to continue growth. FIG. 5(A) shows an immunoblot of total PLB (probed with A1 antibody) in yeast lysates from cells harvested at various times after resuspension in SDglu. PLB protein concentration fell progressively over a 24 hour period to a near zero value. Densiometric analysis of the immunoblot described a mono-exponential relationship, which allowed calculation of half-life for PLB turnover, FIG. 5(B). The half-life of heterologously expressed PLB in S. cerevisiae (BSL1-11B) cells is 111+/−7.5 minutes.

### EXAMPLE 8

Testing the efficacy of vPLB protein: The purpose of this study was to examine whether vPLB protein could remove wt PLB protein from a biological system. To examine this we co-transfected yeast with plasmids for both genes (wtPLB and vPLB), one of which expressed wtPLB from a constitutively active promotor and the other of which expressed vPLB from a galactose dependent promotor. In this way we were able to permit cells to express wtPLB to steady state levels, before they were stimulated to also express vPLB.

The pMAH3 vector contains the promoter and terminator regions of the yeast vacuolar H⁺-ATPase gene (PMA1), which facilitate efficient constitutive expression of heterologous proteins. Once the PLB gene was inserted into the BamH1 cloning site of the pMAH3 vector, the wild type yeast strain BSL1-11B was double transformed with pMAH3:wtPLB and pYES2:pro-vitiate PLB. Different auxotrophic markers on each plasmid allowed simultaneous selection of positive double transformants when grown on uracil, and leucine dropout media.

Introduction of arginine as the N-terminal residue of vitiate PLB is achieved using the method described by Bachmair et al¹⁴. Essentially vitiate PLB is expressed as a ubiquitin fusion protein (pro-vitiate PLB, FIG. 1A), where the C-terminal glycine of ubiquitin is followed by an arginine residue. Pro-vitiate PLB is post translationally de-ubiquitinated by endogenous ubiquitin specific proteases to yield vitiate PLB with an N-terminal arginine.

Since the antibody of choice, A1, recognises both wild type and vitiate PLB, analysis of the expression levels of each species is wholly reliant on the ability to resolve each of them by electrophoresis. The difference in molecular weight of the two species (wild type PLB is 6099, whereas vitiate PLB is processed in cells to a molecular weight of 7128, FIG. 6(i) and (iii)) has been used to achieve this identification. Resolution of these two species (wtPLB and vPLB) by electrophoresis was confirmed by analysis of total protein extracts from yeast expressing each heterologous protein individually. The vectors p MAH3:wtPLB and p YES2:pro-vitiate PLB were transformed separately into BSL1-11B cells. Accordingly, each clone only expressed one protein i.e. either wtPLB or vPLB. These clones were grown in expressing media and total protein lysates were prepared. When proteins were separated by tricine PAGE and analysed by western blotting, the two species were clearly resolved. Vitiate PLB, as expected displayed a slower mobility (FIG. 6(i)) than wild PLB (FIG. 6(ii)). Moreover, when these two lysates were mixed together and analysed clear resolution of the two species was achieved (FIG. 6B(iii)). Confident that the unambiguous identification of wild type PLB and vitiate PLB could be made on the basis of their electrophoretic mobility, the effect of vitiate PLB on the steady state concentration of wtPLB was examined. Yeast transformed with both wtPLB and pro-vitiatePLB vectors were grown to mid log phase at which point cells were harvested, washed twice in SDgal/raf and resuspended to an OD₆₀₀ of 1.0 in SDgal/raf. Cells were sampled over the subsequent 24 hours of growth. Total cell lysate protein was separated by tricine PAGE and analysed by western blotting using an anti-PLB antibody. FIG. 7A(i) shows that high levels of wtPLB are expressed in yeast in the absence of vPLB (FIG. 7A(i) time 0). The steady state concentration of wtPLB falls progressively as vPLB is co-expressed in these cells, to a point at which wtPLB is undetectable (FIG. 7A(i) time 21). Over this same time course, the concentration of vPLB increases. These data were quantified by densitometry and show clearly the increase in vitiate PLB expression and the concomitant decrease in the level of wtPLB, FIG. 7B. Interestingly, it appears that only minimal vPLB expression is required to initiate the reduction in steady state wtPLB. The level of vitiate expression between 2.5-7.5 hours is relatively low, however, the level of wtPLB had already reduced to less than 50% of its original steady state.

### EXAMPLE 9

In order to confirm that the reduction in wtPLB expression level was due to vPLB and not due simply to the change in media conditions (i.e. growth in galctose/raffinose medium), S. cerevisiae were transformed with the pMAH3:wtPLB vector and the pYES2 vector containing no insert (i.e. no vitiate PLB gene). Cells constitutively expressing wtPLB were transferred to SDgalactose/raffinose media to replicate the expressing conditions of the previous experiment. FIG. 7A(ii) shows that in the absence of vitiate PLB the steady state level of wtPLB is unaffected by growth on a galactose/raffinose carbon source.

The results are consistent with the catalytic removal of wtPLB in cells expressing vitiate PLB as predicted in FIG. 2. The present invention therefore provides molecules and methods for replacing wtPLB with a PLB molecule of our design (vPLB) which might prove advantageous in the treatment of certain cardiac conditions. Indeed vPLB may be further modified as hereinbefore described to include mutations which affect ability of PLB to control Ca2+-pump. It is envisaged that use of these in combination with vPLB sequence would be useful in gene therapy.

### EXAMPLE 10

Testing the efficacy of vPLB protein. The purpose of this study was to examine whether vPLB protein could remove wt PLB protein from a biological system and to therefore confirm the results of Example 8. Example 10 demonstrates that o-expression of Vitiate PLB reduces the steady state level of wtPLB.

Both wtPLB and vitiatePLB are recognized by the α-PLB antibody, A1 and, as a result, previous examples, such as Example 8 and 9, have utilized the difference in electrophoretic mobility to distinguish the two molecules. In contrast, in the following example, the A1 epitope of the vitiate PLB was removed and a c-Myc epitope tag was engineered on the C-terminus of the molecule. Both pMAH3:wtPLB and pYES2: vitiatePLB-c-Myc were co-transformed into S. cerevisiae (using BSL1-11B cells). The auxotrophic markers leucine and uracil were used to select positive transformants. The positive transformants were then grown on minimal glucose media, allowing the constitutive expression of wtPLB. The vitiate PLB-cmyc was placed under the control of a switchable promoter. Once cells had reached steady state wtPLB expression, vitiate PLB-cmyc expression was turned on. At the mid log phase of growth, cells were switched to a galactose and raffinose based minimal media. An aliquot of cells was removed at time zero and further aliquots were removed after 8 hours of co-expression. The samples were analysed for the presence of both wtPLB and vitiatePLB-cmyc.

The A1 Western blot (FIG. 8) shows that, at time zero, the yeast maintain a robust steady state level of wtPLB expression. However, after 8 hours of co-expression with vitiatePLB-cmyc, the expression level has been reduced to undetectable levels. In contrast, the level of vitiatePLB-cmyc reciprocates this pattern of expression: at time zero, the level of expression of vitiatePLB-cmyc is undetectable, whilst after 8 hours of co-expression, a robust steady state expression is observed. This confirms the results given in previous Examples such as Example 8.

In the present invention we have “designed” a molecule (vPLB nucleic acid or the protein encoded thereby or the vPLB protein itself) based on the principles governing protein degradation of the N-end rule and also the propensity of proteins (PLB) to oligomerise.

We have designed a vPLB nucleic acid incorporating a destabilising N-terminal residue encoding arginine with the absence of amino acids encoding a lysine residue which encodes a vPLB protein. Thus we are able to reduced a modified/mutated form of PLB protein which can form mixed oligomers with wtPLB and stimulate, due to its destabilising N-terminal residue and lack of ubiquitination potential. Since vPLB protein cannot be ubiquitinated itself, it is able to direct the degradation machinery to its oligomer partners. Thus, partner molecules are be tagged for destruction by a process of trans-ubiquitination provoked by vPLB protein.

The yeast S. cerevisiae is null for PLB and therefore provides a suitable genetic background to test the vPLB hypothesis. The steady state expression of PLB protein that is observed in cardiac myocytes was mimicked in this system by driving heterologous expression of wtPLB using a constitutive promoter. A robust steady state level of wtPLB protein expression was observed. Subsequent induction of vitiate PLB expression resulted in a dramatic decline in the steady state level of wtPLB. Thus clearly demonstrating that vitiate PLB was able to capture wtPLB (through formation of an oligomeric species) and target it, by trans-ubiquitination, to the degradation machinery. In control experiments double transformants were engineered containing a p YES2 vector without the cDNA encoding for vitiate PLB. Transfer of cells to expressing media (SDgal/raf) resulted in no change in the steady state level of wtPLB. This suggests that the decline in wtPLB steady state is wholly reliant on the expression of vitiate PLB and that vitiate PLB is able to provoke the rapid removal of wtPLB from S. cerevisiae. We can therefore draw two important conclusions, firstly, heterologous PLB is able to oligomerise adding further credence to the idea that under native conditions PLB does indeed have the propensity to form an oligomer, most likely a pentamer. Secondly, by introducing the appropriate signal motifs we can force the degradation machinery to trans-ubiquitinate.

The present invention has important implications for correcting low Ca²⁺pump rates and therefore SR dysfunction in cardiac myocytes. It is envisaged that introduction of vPLB nucleic acid or the protein encoded thereby or the vPLB protein itself, in cardiac myocytes would stimulate the catalytic replacement of endogenous PLB.

In the present invention we have provided a means to replace PLB with a designer vPLB molecule be it in the nucleic acid or protein form. These entities provide enormous flexibility. Using vPLB nucleic acid or the protein encoded thereby or vPLB protein itself could potentially control the steady state level of endogenous PLB. This is achieved by altering the N-terminal residue of vPLB, which would exert its effects by changing the rate of wt or endogenous PLB degradation. The steady state level of a protein is determined by the rate of synthesis and the rate of protein degradation. One mechanism of altering the steady state level of a protein is to alter the rate of degradation. The transient increase in many transcription factors is controlled in this way. In the example of vPLB the rate of degradation of the target (in this case wtPLB) is determined by the N-terminal arginine of vPLB. The rate of degradation is rapid since arginine is a destablising N-terminal residue. In this way vPLB is able to stimulate a dramatic decline in the steady state level of wtPLB. However, replacing the N-terminal arginine of vPLB with a more stabilising residue would reduce the steady state level of wtPLB to some intermediate level. In this way vPLB may allow us to control the steady state level of wtPLB or indeed any target protein.

### REFERENCES

1. Cong F, Zhang J, Pao W, Zhou P, Varmus H. A protein knockdown strategy to study the function of beta-catenin in tumorigenesis. BMC Mol Biol. 2003; 4:10
2. Minamisawa S, et al Chronic phospholamban-sarcoplasmic reticulum calcium ATPase interaction is the critical calcium cycling defect in dilated cardiomyopathy. Cell. October 29, 1999; 99(3):313-22.
3. Hoshijima M et al Chronic suppression of heart-failure progression by a pseudophosphorylated mutant of phospholamban via in vivo cardiac rAAV gene delivery. Nat Med. 2002 August; 8(8):864-71
4. Schmitt J P et al Dilated cardiomyopathy and heart failure caused by a mutation in phospholamban Science. February 28, 2003; 299(5611):1410-3.
5. Haghighi K et al. Human phospholamban null results in lethal dilated cardiomyopathy revealing a critical difference between mouse and human. J Clin Invest. 2003 March; 111(6):869-76.
6. Johnson E S, Gonda D K and Varshavsky A. Cis-trans recognition and subunit-specific degradation of short-lived proteins. Nature. 1990, 346:287-91.
7. Gietz, R. D. and R. A. Woods. Transformation Of Yeast By The LiAc/SS Carrier DNA/PEG Method. Methods in Enzymology 2002 350: 87-96.
8. Smith, P. K. et al. Measurement of Protein Using Bicinchoninic Acid. Anal. Biochem. 1985, 150: 76-85
9. Suzuki T, Wang J H. Stimulation of bovine cardiac sarcoplasmic reticulum Ca2+ pump and blocking of phospholamban phosphorylation and dephosphorylation by a phospholamban monoclonal antibody. J Biol Chem. May 25, 1986; 261(15):7018-23
10. Davis B G, Sala R F, Hodgson D R, et al. Selective Protein Degradation by Ligand-Targeted Enzymes: Towards the Creation of Catalytic Antagonists. Chembiochem. 2003; 4:533-537
11. Varshavsky A. The N-end rule: functions, mysteries, uses. Proc Natl Acad Sci USA. 1996, 93:12142-9.
12. Song J et al Regulation of membrane and subunit interactions by N-myristoylation of a G protein alpha subunit in yeast. J Biol Chem. 1996 271:20273-83
13. Powell K S, Latterich M. The making and breaking of the endoplasmic reticulum. Traffic. 2000 September; 1(9):689-94
14. Bachmair A, Finley D, Varshavsky A. In vivo half-life of a protein is a function of its amino-terminal residue. Science. 1986, 234:179-86
15. Kovacs R J, Nelson M T, Simmerman H K, Jones L R. Phospholamban forms Ca2+-selective channels in lipid bilayers. J Biol Chem. 1988, 263:18364-8.
16. Bowles D J, Marcus S E, Pappin D J, Findlay J B, Eliopoulos E, Maycox P R, Burgess J. Posttranslational processing of concanavalin A precursors in jackbean cotyledons. J Cell Biol. 1986, 102:1284-97
17. Mendez E, Salas-Ocampo M P, Munguia M E, Arias C F. Protein products of the open reading frames encoding nonstructural proteins of human astrovirus serotype 8. J Virol. 2003, 77:11378-84.

1. A method of controlling levels/concentrations of a target moiety comprising:
(i) introducing into a cell a product comprising at least one of each of the following modules: a targeting module, a destruction module and a replacement module;
(ii) targeting the target moiety with the targeting module of the product so as to bind them together or at least bring the target moiety into close proximity with the product so that the destruction module can effect degradation of the target moiety; and
(iii) replacing the target moiety with a replacement module, the replacement module either being a modified form of the target moiety itself or a functional unit which restores normal metabolic activity of the cell.

2. The method according to claim 1 wherein the target moiety is a protein.

3. The method according to claim 1 wherein the targeting module is selected from the group consisting of a protein, polypeptide, aptamer of and drug-like molecule or portions or fragments thereof.

4. The method according to claim 1 wherein the replacement module of the product is in the form of a modified form of the target moiety itself or is a functional unit which is capable of restoring normal metabolic activity of the cell into which the product has been introduced.

5. The method according to claim 1 wherein the destruction module acts directly or indirectly on the target moiety.

6. The method according to claim 5 wherein the destruction module acts directly on the target moiety and the destruction module comprises a proteinase.

7. The method according to claim 5 wherein the destruction module acts indirectly on the target moiety and the destruction module comprises a mutation or deletion such that it alters degradation of the target moiety.

8. The method according to any preceding claim 1 wherein the replacement module is modified to retain at least some of the beneficial attributes and to remove/delete at least some of the adverse attributes of the target moiety.

9. A product comprising at least one of a targeting module of claim 3, a destruction module of claim 5 and a replacement module of claim 1.

10. A product comprising a targeting module comprising a β-catenin binding domain of E-cadherin, a destruction module comprising an F-box mutated such that it is not able to bind to the phosphorylated form of β-catenin and a replacement module comprising wild type β-catenin.

11. A product comprising a targeting module comprising an inhibitor of alcohol dehydrogenase (ADH), a destruction module comprising a protease domain and a replacement module comprising a functional ADH with any protease recognition motifs removed and specific mutations to substantially reduce sensitivity to a targeting module.

12. A product comprising a targeting module comprising a PLB target motif, a destruction module comprising absence of either or both lysine 3 and/or 27 so that ubiquitination cannot occur together with an N-terminal domain exhibiting a destabilising N-terminal residue and a replacement module comprising a modified PLB sequence such that it is unable to inhibit Ca2+-pump activity.

13. An isolated nucleic acid comprising a modified or mutated form of a PLB nucleic acid sequence (vPLB) comprising at least one modification or mutation in the N-terminal domain and/or a deleted or mutated region that encodes a lysine residue in the expressed protein so that at least one lysine is not expressed in the protein encoded by the modified or mutated vPLB nucleic acid sequence.

14. Nucleic The nucleic acid according to claim 13 wherein the deleted or mutated region of the vPLB affects lysine 3 residue and/or lysine 27 so that either or both of lysine 3 and 27 is/are not expressed in the protein encoded by the vPLB nucleic acid.

15. The nucleic acid according to claim 13 wherein modification or mutation of the 5′ end of the nucleic acid polymer encoding PLB is such that the N-terminal domain of the protein comprises replacement of the region encoding a methionine with an unstructured linker sequence.

16. The nucleic acid according to claim 15 wherein the linker sequence comprises a nucleic acid sequence encoding at least one glycine residue.

17. The nucleic acid according to claim 15 wherein the linker sequence further comprises an N-terminal residue of arginine residue or another destabilising residue.

18. The nucleic acid according to claim 15 wherein the linker sequence comprises a nucleic acid sequence encoding between 1-1000 residues or between 1-100 or between 1-50 glycine residues.

19. The nucleic acid according to claim 15 wherein the linker sequence comprises a nucleic acid sequence encoding between 5-35 glycine residues.

20. The nucleic acid according to claim 13 further comprising any one or more of the mutations resulting in altered expression of a selected amino acid selected from the group consisting of N34A, L31A, E2A, L42A, I38A, L7A, F35A, I12A, R14A, V4A, I48A, R9A, L52A, P21A, V49A, R25A, Q26A, R13A, L28A, L39, P21A and A24V.

21. A protein encoded by the vPLB nucleic acid according to claim 13.

22. A vector comprising the product of claim 9 or the nucleic acid of claim 13.

23. The vector according to claim 22 further comprising a promoter for driving expression of the product or nucleic acid.

24. A host cell transformed with the vector of claim 22.

25. Use of a vPLB nucleic acid claim 13 or the protein encoded thereby as a pharmaceutical.

26. A method of producing a vPLB nucleic acid molecule comprising:
(i) modifying or mutating native or natural PLB so that a region encoding of a methionine residue at the N-terminus of the protein is replaced with an linker sequence comprising a sequence of nucleic acids encoding at least one glycine residue and at least one arginine residue or other destabilising residue at the N-terminus of the vPLB protein; and
(ii) deleting or mutating a region of nucleic acids that encode at least one or both lysine residue(s) from the original PLB sequence so that lysine(s) is/are not expressed in the protein at a location to participate in N-end rule directed ubiquitination.

27. The method according to claim 26 further comprising modifying the vPLB so as to retain at least some beneficial qualities and to reduce at least some considered detrimental.

28. The method according to claim 26 further comprising engineering mutations resulting in altered expression of a selected amino acid selected from the group consisting of N34A, L31A E2A, L42A, I38A, L7A, F35A, I12A, R14A, V4A, I48A, R9A, L52A, P21A, V49A, R25A, Q26A, R13A, L28A, L39A, P21A and A24V.

29. The method of treating cardiac disorders selected from the group consisting of acute congestive heart failure precipitated by mycardial ischemia, hypertrophic cardiomyopathy and dilated cardiomyopathy, which have a common feature of diastolic dysfunction and lethargic Ca²⁺ handling by the sarcoplasmic reticulum (SR) comprising administering a therapeutically effective amount of vPLB nucleic acid according to claim 13 or protein encoded thereby or a vector comprising the nucleic acid or a protein expressed by such nucleic acid, to a subject in need of treatment for the specified conditions.
